(19) United States
(12) Patent Application Publication
(10) Pub. No.: US 2014/0046332 A1
(43) Pub. Date: Feb. 13, 2014

(54) STOPPING TOOL GUIDES AND COMBINATIONS THEREOF WITH SURGICAL GUIDES, METHODS FOR MANUFACTURING AND USES THEREOF

(71) Applicants: Ayishwariya Premanathan, Ampang (MY); Louis James Keppler, Leuven (BE)

(72) Inventors: Ayishwariya Premanathan, Ampang (MY); Louis James Keppler, Leuven (BE)

(21) Appl. No.: 14/113,471

(22) PCT Filed: Apr. 27, 2012
(86) PCT No.: PCT/EP12/57719

§ 371 Date: Oct. 23, 2013

Related U.S. Application Data
(60) Provisional application No. 61/479,963, filed on Apr. 28, 2011.

(30) Foreign Application Priority Data
Jun. 29, 2011 (EP) 11171837.5

(51) Int. Cl. A61B 17/15

(52) U.S. Cl. CPC A61B 17/151
USPC 606/87; 29/592

(57) ABSTRACT

The present invention relates to stopping tool guides for use during surgical interventions and combinations of these stopping tool guides with surgical guides. The present invention further relates to methods for the manufacture of such stopping tool guides and combinations thereof with surgical guides. The stopping tool guides and combinations of such stopping tool guides with surgical guides according to the present invention are particularly useful in surgical osteotomy.

### FIELD OF THE INVENTION

The present invention relates to stopping tool guides for use during surgical interventions and combinations of these stopping tool guides with surgical guides. The present invention further relates to methods for the manufacture of such stopping tool guides and combinations thereof with surgical guides.

### BACKGROUND

A common procedure in orthopaedic surgery is an osteotomy wherein a bone is partially cut or wherein a segment of the bone is removed, followed by repositioning of the bone. Typical examples are tibial and femoral osteotomies. Various surgical guides exist for such surgical interventions, which the surgeon can use to accurately transfer a pre-operative plan into the operating room with wide applications in orthopaedic surgery. These guides may guide a cutting instrument along a pre-defined cutting path; the cutting paths most commonly refer to planar cuts but can also contain more than one plane. For a satisfactory outcome of the surgical procedure, it is essential that the cutting instrument is guided along the correct cutting path, but it is also crucial that the surgeon stops cutting at the correct position along the cutting path. Usually, this is done by freehand positioning of stopping tools.

In recent years, with the advent of locking plates, fixation techniques have improved significantly but the correct planning and execution of the operation remains difficult. Despite the availability of CT and MRI 3D imaging, surgical planning is still traditionally performed on 2D plain X-rays. Especially in case of multi-planar deformities, accounting for the majority of cases, this technique is obsolete and prone to error. In addition, the traditional intra-operative tools such as rulers and protractors for checking the achieved geometrical correction are crude and inaccurate. Accordingly, undertaking an osteotomy procedure is technically demanding and carries specific risks of neurovascular injury through incorrect planning and execution, and insufficient fixation of the used devices or guides.

US patent application US 2009/0571969 discloses a patient-specific alignment guide comprising a cutting surface and a laser indicator. However, this does not offer a mechanical stop for the cutting instrument.

US patent application US 2005/0075641 discloses an apparatus and procedure for surgical correction of joint alignment. In the application, it is suggested to leave a drill in a drill hole during cutting of the osteotomy, wherein the drill acts as a shield to limit the depth of the cut. However, such a solution is often impractical.

U.S. Pat. No. 4,627,425 discloses an osteotomy appliance for making a pair of intersecting cuts in a bone. Before making the cuts, a pin is inserted approximately at the intersection of the two cuts to be made. However, this method has a limited accuracy.

US patent application US 2008/147073 discloses an apparatus for performing an osteotomy cut into the upper portion of the tibia, comprising a cutting slot and a stopping tool. However, the apparatus is bulky and therefore impractical to use.

Accordingly, there is a need for improved means which allow accurate guiding of a surgical cutting tool, and stopping the surgical cutting tool at the correct position. The present invention provides an innovative solution that uses guide technology to achieve this aim. The surgical instruments of the present invention are easy to use and offer a more accurate positioning of a stopping tool, thus increasing the chances of a successful osteotomy.

### SUMMARY OF THE INVENTION

The present invention relates to stopping tool guides and combinations of such stopping tool guides with surgical guides for use in orthopaedic surgery, and more particularly for use in surgical osteotomy.

The stopping tool guides according to the present invention allow to correctly and accurately execute bone osteotomies with the aid of (patient-specific) surgical guides and stopping tools based on virtual pre-operative three-dimensional images and planning. Since the orientation of the surgical guide with regard to the stopping tool guide in the combinations of the invention is fixed, the present invention allows for simple, pre-determined and accurate positioning of the stopping tool guides in combination with surgical guides onto the patient's bone without any necessity for additional adjustments or further fine-tuning.

Also, the stopping tool guides of the present invention and combinations thereof with surgical guides allow for minimally invasive surgery as the stopping tool can in most cases be inserted percutaneously and the need for an additional surgical intervention to define the position of the stopping tool is eliminated.

In a first aspect, the present invention provides a combination of a stopping tool guide and a surgical guide for a cutting instrument for cutting a bone of a patient, wherein said surgical guide comprises:

(i) at least one guiding component for guiding said cutting instrument along a plane,
(ii) at least one surface structure for contacting said patient's bone, and
(iii) a connecting element for connecting said surgical guide to said stopping tool guide; and wherein said stopping tool guide comprises:
(i) a connecting element for connecting the stopping tool guide to said connecting element of said surgical guide, whereby the stopping tool guide is reversibly detachable from the surgical guide,
(ii) at least one sleeve element for guiding said stopping tool in orientation direction y, and
(iii) a bridge element interconnecting said connecting element with said sleeve element,

wherein the angular difference that is bridged by said bridge element between said connecting element and said sleeve element is between 30° and 180°, and

wherein said orientation direction y of said sleeve element crosses said plane for guiding said cutting instrument, such that said stopping tool functions to prevent said cutting instrument, when placed in said guiding component, from advancing beyond a planned or predetermined depth. In particular embodiments said direction of said sleeve element lies within the plane for guiding said cutting instrument.

In particular embodiments of the present invention, the bridge element of the stopping tool guides of the invention ensures that said sleeve element is not in contact with said surgical guide and/or with the bone upon placement of said combination of said stopping tool guide and said surgical guide onto the bone.

In certain particular embodiments of the present invention, the sleeve element of the stopping tool guides of the invention is reversibly detachable from said stopping tool guide. In some embodiments, the sleeve element comprises a reversibly detachable sleeve insertion element, which is reversibly inserted into said sleeve element in said direction y. In certain embodiments, the sleeve element further can be provided with a drill sleeve for guiding a drilling instrument.

In particular embodiments of the invention, the surgical guide further comprises an insert feature on the surface structure of the surgical guide for guiding said stopping tool into the bone in orientation direction y.

In certain particular embodiments, the surgical guide is a patient-specific surgical guide.

In a further aspect, the present invention provides a stopping tool guide comprising:

(i) a connecting element for connecting the stopping tool guide to a surgical guide,
(ii) a sleeve element for guiding said stopping tool in orientation direction y, and
(iii) a bridge element interconnecting said connecting element with said sleeve element,

wherein the angular difference that is bridged by said bridge element between said connecting element and said sleeve element is between 30° and 180°.

In particular embodiments of the invention, the bridge element of the stopping tool guide ensures that said sleeve element of said stopping tool guide is not in contact with said surgical guide and/or with the bone upon placement of the said stopping tool guide onto the a surgical guide or onto a bone.

In a further aspect, methods for manufacturing a combination of a stopping tool guide and a surgical guide as described herein are provided, said methods comprising:
(a) obtaining an image of the bone,
(b) determining the appropriate position with regard to the bone of said guiding component for guiding a cutting instrument along a plane of said surgical guide, based on the pre-operative planning of the desired path of said cutting instrument into the bone;
(c) determining the appropriate position of a stopping tool in the bone, based on the pre-operative planning of the desired path of said cutting instrument into the bone;
(d) designing the combination of said stopping tool guide and said surgical guide based thereon such that:
   said guiding component for guiding a cutting instrument, upon placement of the surgical guide on the bone, is positioned as determined in step (b), and
   said sleeve element of said stopping tool guide ensures the correct orientation of the stopping tool upon placement of the stopping tool guide on the bone,
   said bridge element ensures that said stopping tool can be connected to said surgical guide such that the angular difference that is bridged by said bridge element between said connecting element and said sleeve element is between 30° and 180°, and
   ensures that said sleeve element is not in contact with said surgical guide and/or with the bone upon placement of said combination of said stopping tool guide and said surgical guide onto the bone, and
      (e) manufacturing said stopping tool guide and said surgical guide.

In a further aspect, the methods for performing accurate surgical osteotomy on a bone are provided, which methods comprise:

(a) providing a combination of a stopping tool guide and a surgical guide as described herein,
(b) securing said combination of said stopping tool guide and said surgical guide onto the bone by aligning it in the desired direction with regard to the anatomical surface on the bone,
(c) introducing a stopping tool through the sleeve element of said stopping tool guide into the bone, and
(d) performing a surgical osteotomy by introducing a cutting instrument through said at least one guiding component of said surgical guide, whereby said cutting instrument is stopped by said stopping tool.

### BRIEF DESCRIPTION OF THE DRAWINGS

The following figures of specific embodiments of the invention are merely exemplary in nature and are not intended to limit the present teachings, their application or uses. Throughout the drawings, corresponding reference numerals indicate like or corresponding parts and features.

FIG. 1 represents the combination of a stopping tool guide and a surgical guide according to a specific embodiment of the present invention viewed from different angles (A, B, C).

FIG. 2 represents a detailed illustration of the sleeve element according to a specific embodiment of the present invention.

FIG. 3 illustrates a bone having an inserted stopping tool, with (A) and without (B) the surgical guide.

FIG. 4 represents an illustration of the sleeve element according to a specific embodiment of the present invention.

FIG. 5 illustrates two connecting elements and a locking pin according to a particular embodiment of the present invention, each independently (A) and when connected (B, C).

FIGS. 6 A and B each represent a stopping tool guide according to a particular embodiment of the present invention.

List of reference numerals used in the Figures. Each of these illustrations represents particular embodiments of the features concerned and the corresponding features are not to be interpreted as limited to this specific embodiment.
(1) anatomical part of a patient
(2) surgical guide
(3) guiding component for guiding a cutting instrument
(4) connecting element for connecting surgical guide to stopping tool guide
(5) stopping tool guide
(6) connecting element for connecting the stopping tool guide to the connecting element of the surgical guide
(7) bridge element
(8) sleeve element
(9) sleeve insertion element
(10) stopping tool
(11) insert feature
(12) partial bone osteotomy
(13) hole for locking pin
(14) locking pin

### DETAILED DESCRIPTION

The present invention will be described with respect to particular embodiments but the invention is not limited thereto but only by the claims. Any reference signs in the claims shall not be construed as limiting the scope thereof.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the embodiments disclosed herein. The terms or definitions used herein are provided solely to aid in the understanding of the embodiments disclosed herein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the envisaged scope, and form different embodiments, as would be understood by those in the art. For example, in the following claims, any of the claimed embodiments can be used in any combination.

Particular embodiments of the present invention provide stopping tool guides (hereinafter also referred to as “stopping tool guides of the (present) invention”) and combinations of such stopping tool guides with surgical guides (hereinafter also referred to as “combinations of the (present) invention”) for use in orthopaedic surgery, and more particularly for use in surgical osteotomy.

The stopping tool guides as described herein allow to correctly and accurately execute bone osteotomies with the aid of (patient-specific) surgical guides and stopping tools based on virtual pre-operative three-dimensional images and planning. More particularly, in case of a guide for a cutting tool, while the surgical guide will determine the location and orientation of the incision, the stopping tool guide make it possible to determine the maximal depth of the incision.

As the (relative) positions of the stopping tool guide and the surgical guide are based on pre-operative imaging and planning of a specific part of the body of a patient that is to be treated, these positions need not to be adjusted anymore during the surgical intervention but can be determined accurately and precisely on the forehand, i.e. during the designing process. Indeed, as opposed to the prior art devices, the devices and combinations described herein do not have intra-operative movable and adjustable parts, which inevitably result in a higher risk for positioning discrepancies and errors during surgery. Accordingly, since the orientation of the surgical guide with regard to the stopping tool guide in the combinations presently claimed is fixed, the embodiments described herein allow for simple, pre-determined and accurate positioning of the stopping tool guides in combination with surgical guides onto the patient's bone without any necessity for additional adjustments or further fine-tuning.

In addition, the stopping tool guides and combinations thereof with surgical guides, are compatible with minimally invasive surgery because the stopping tool can in most cases be inserted percutaneously and the need for an additional incision or surgical intervention to define the position of the stopping tool is therefore eliminated. This results in a shorter hospitalization time and reduces the risk for complications, the pain and the formation of scars for the patient.

The surgical guides, stopping tool guides and combinations thereof claimed herein each or separately can be patient-specific devices which allow accurate and stable introduction of a surgical instrument into the bone of a patient. More particularly, these surgical guides, stopping tool guides and combinations thereof can include medical-image-based patient-specific surgical guides, stopping tool guides and combinations thereof providing the ability to accurately insert a surgical instrument into the patient's bone according to a predefined (pre-operatively determined) planning.

Accordingly, the term “patient-specific” as used herein refers to the surgical, therapeutic or diagnostic devices, tools, implants or guides as described herein, which are designed starting from an individual patient's anatomy to provide patient-specific devices, tools, implants or guides having a custom fit or functioning in a unique, customized manner for a particular individual patient. The use of patient-specific devices, tools, implants or guides allows for improved or optimized surgical interventions, orthopaedic structures and/or kinematics for the patient. Similar benefits are obtained when such patient-specific devices are used in combination with standard implants, tools, devices, surgical procedures, and/or other methods.

Accordingly, in the context of the embodiments of the present invention, the term “patient-specific” is used to describe a custom device, tool, implant or guide that is specific to the individual patient's anatomy. Therefore, such a device, tool, implant or guide is provided with at least one surface that is conform or complementary with at least part of the patient's anatomy. Such a complementary surface is also referred herein as a “patient-specific area”.

Thus, in a first aspect, a combination of a stopping tool guide and a surgical guide is provided. More particularly, the surgical guide is a guide for a cutting instrument, for cutting in a bone of a patient, e.g. in the context of an osteotomy. A surgical guide suitable for use as envisaged herein at least comprises:

(i) at least one guiding component for guiding the surgical instrument, more particularly the cutting instrument along a plane,
(ii) at least one surface structure, and
(iii) a connecting element for connecting the surgical guide to the stopping tool guide.

The surgical guides that are used in combination with the stopping tool guides of the present invention at least comprise a guiding component, a surface structure, and a connecting element for connecting the surgical guide to a stopping tool guide. The different components of the surgical guides suitable for use in the combinations of the present invention are described more in detail hereafter. Unless specified (e.g. in the context of placement of the tools of the invention), reference to the position of the bone is intended to refer to the position of the bone when the surgical guide has been placed onto the bone.

In particular embodiments of the present invention, where the stopping tool guides of the invention and combinations thereof with surgical guides are used for performing accurate surgical osteotomy, the surgical instrument is a cutting instrument for cutting into a bone of a patient. Examples of cutting instruments include a knife, saw, jig saw or the like. In these particular embodiments, the guiding component may contain at least a (narrow) slot or flat surface, the position and orientation of which will correspond to the pre-operatively planned cutting plane, i.e. the plane along which the cutting instrument is guided by the guiding component. The dimensions of the slot or surface will be such to allow passage or suitable guidance of the knife, saw, jig saw or the like that is used.

Typically, the guiding component is positioned on the surface structure (as further described herein) of the surgical guide engaging with the patient's bone anatomy, such that a surgical instrument which is passed through the guiding component can be inserted into the bone at a desired location. The guiding component can be positioned in any direction with regard to the bone as long as it provides access to a surgical instrument for reaching the bone at the desired location.

The surgical guides that are used in combination with the stopping tool guides of the present invention further comprise at least one surface structure through which the guide makes contact with the patient's bone.

In particular embodiments of the invention, such a surface structure may consist of one or more parts and may comprise a patient-specific component. Accordingly, in case a patient-specific component is present in the surgical guides of the invention, the surgical guides are referred to herein as “patient-specific surgical guides” and the at least one surface structure functions so as to ensure the correct placement of the guide tool on the patient's bone. Accordingly, the at least one surface structure of the patient-specific surgical guides of the invention comprises a patient-specific component comprising one or more patient-specific areas and optionally comprises one or more non-patient specific areas. A patient-specific area is an area on the inside of the surface structure which contacts and interlocks with the bone on which the guide is to be placed in a patient-specific way. Typically, a patient-specific area is a surface area that is fully complementary to an anatomical surface of/on the bone. In particular embodiments, this complementarity allows a clearance between the at least one surface of the guide and the patient's bone of between 0.1-0.5 mm, more particularly between 0.1-0.2 mm. The one or more patient-specific areas are positioned on the inside of the surface structure such that they can be contacted with/positioned opposite to the corresponding surface of the bone upon placement of the guide. When a patient-specific area of the surface structure is contacted with/positioned opposite to its corresponding complementary part of the surface of the bone the two surfaces can interlock, fixing the surface structure (and thus the guide) into a predetermined position onto the bone.

Thus, the patient-specific guides according to the present invention comprise at least one patient-specific area, which is typically a patient-specific surface that is designed to fit onto at least part of the bone of a patient and that is fully complementary to sufficient features of that part of the bone to determine a unique locking position. Typically, a patient-specific surface is selected based on anatomical features present on the bone. However, it can be envisioned that in particular embodiments features are introduced onto the patient's bone to allow the generation of patient-specific area based thereon.

The size of the one or more patient-specific areas of the at least one surface structure of the surgical guides of the present invention is not critical. Depending on the embodiment they can span all or part of the inside of the surface structure.

The location of the one or more patient-specific areas in the surface structure is not critical and may be spread out over one or more parts of the surface.

In particular embodiments, the patient-specific surgical guide as described herein further comprises a fitting tool for use in a method for validating the correct position of the surgical guide on the patient's anatomy, as described in patent application U.S. Ser. No. 13/106,448, which is hereby incorporated by reference.

The surgical guides that are used in combination with stopping tool guides as described herein further comprise a connecting element. Such a connecting element directly or indirectly connects the surgical guide to the stopping tool guide of the invention. The connecting element ensures that the surface structure of the surgical guide (as described herein) and the bridge element of the stopping tool guide (as further described herein) are interconnected. In particular embodiments, the orientation of the stopping tool guide with regard to the surgical guide is (at least partially) determined by the position and orientation of the connecting element on the surgical guide and/or the nature of the bridge element. Where the bridge element is a generic feature the orientation of the stopping tool can be adjusted by the position and orientation of the connecting element on the surgical guide.

The connecting element may in certain embodiments comprise one or more connecting subparts. A connecting element may for example be a clip-on mechanism or clip feature or any type of suitable locking mechanism.

Similarly, the stopping tool guides that are used in combination with a surgical guide in particular embodiments at least comprise a connecting element for connecting the stopping tool guide to the connecting element of the surgical guide (as described herein), a sleeve element (for guiding the stopping tool) and a bridge element (which allows accurate position and orientation of the sleeve element relative to the guiding tool). The different components of the stopping tool guides of the present invention are described more in detail hereafter. Unless specified (e.g. in the context of placement of the tools of the invention), reference to the position of the bone is intended to refer to the position of the bone when the stopping tool guide has been placed onto the bone.

The connecting element of the stopping tool guide according to the invention may in certain embodiments comprise one or more connecting subparts, which may or may not be directly interconnected. A connecting element may for example be a clip-on mechanism or clip feature but may well be any type of suitable locking mechanism.

In particular embodiments the connecting element of the guiding tool and the connecting element of the stopping tool interconnect by the presence of complementary features or interlocking mechanisms such as pegs/holes, snap-fits, etc.

Additionally or alternatively, the connecting element of the guiding tool and the connecting element of the stopping tool may each comprise one or more (optionally threaded) holes which allow insertion of one or more locking pins and/or locking screws. The use of locking pins and/or screws allows a reversible locking of the stopping tool guide onto the surgical guide. The locking pin(s) and/or screw(s) may be standard elements or may be designed specifically for the connecting elements. In further embodiments, the combination of the stopping tool guide and surgical guide as described herein further comprises one or more locking pins and/or screws.

Additionally or alternatively, a first connecting element (which may be located on the stopping tool guide or on the surgical guide) comprises a lever element with a hinge, whereby the lever element may snap on a second connecting element (which, depending on the location of the first connecting element, may be located on the surgical guide or the stopping tool guide), thereby locking the relative position of the connecting elements (and consequently the stopping tool guide and the surgical guide). In further embodiments, at least a part of the lever is shaped to engage with a matching feature such as a recess or protrusion present on the second connecting element. In particular embodiments, the lever makes a sound, such as a clicking sound, when it snaps on the second connecting element, thus allowing an auditory verification of whether the stopping tool guide and the surgical guide are connected.

The concept of the present invention is that a stopping tool, which is used to prevent the surgical instrument from penetrating the bone beyond a predetermined depth, is mounted onto the surgical guide, by way of the interaction of the connecting elements on the stopping tool guide and surgical guide respectively. More particularly a stopping tool guide, comprising a sleeve for fitting a stopping tool, determines the position and orientation of the stopping tool. The stopping tool guide is positioned such that a stopping tool, when inserted into the sleeve, crosses the cutting or boring path of a surgical instrument introduced through the guiding component of the surgical guide. The connection of the connecting elements is ensured at a specific position on the surgical guiding tool. When the guiding tool is placed on the bone, this corresponds to a specific position (first area) relative to the central axis of the bone. By way of the bridge element, the stopping tool placed on the surgical guide extends to another area of the bone such that the sleeve of the stopping tool is positioned at an angle relative to the first area of the bone (the angle being determined based on the central axis of the bone).

The relative position of the interacting connecting elements and the position of the sleeve of the stopping tool can be expressed by an angle relative to the central axis of the bone. It will be understood that the connecting elements may but need not necessarily extend perpendicularly from the surface of the bone (as is typically the case where the connecting elements are specifically fitting protrusions and sleeves). Nevertheless, for purposes of clarity, reference will be made to the orientation of the connecting element. This corresponds to the orientation of the central axis of the connecting element which is perpendicular to the surface of the bone (and or surgical guide). Typically, in this context, when referring to the connecting element, the feature ensuring interconnection between the connecting element on the surgical guide and the stopping tool guide is intended.

In particular embodiments of the present invention, the connecting element is said to have an orientation direction x. In such embodiments, orientation direction x can be but is not limited to the direction in which the connecting element of the stopping tool guide is positioned on the surgical guide; this is particularly the case where the connecting element of the stopping tool guide is placed onto the surgical guide in a direction that is not parallel (but for example perpendicular or substantially perpendicular) to the central longitudinal axis of the bone, e.g. by using a slide-on or clip-on mechanism. However, in certain other particular embodiments, where the connecting feature of the stopping tool guide is placed onto the surgical guide in a direction that is parallel or substantially parallel to the central longitudinal axis of the bone, e.g. by a slide lock mechanism, orientation direction x is different from the direction in which the stopping tool guide is placed or connected to the surgical guide. In further particular embodiments, orientation direction x corresponds to the central (i.e. symmetry or rotational axis) of the connecting element (and/or the feature ensuring connection of the connecting element of the surgical guide with that of the stopping tool guide). For instance when the connecting element of the guiding tool (and the corresponding connecting element of the stopping tool guide) has a cylindrical shape extending perpendicularly from the surface of the guide (or bone), orientation direction x corresponds to the central longitudinal axis of the cylindrical shape of the connecting element.

The stopping tool guides according to the present invention that can be used in combination with surgical guides further at least comprise a sleeve element for guiding said stopping tool in orientation direction y.

The sleeve element contains at least one means of guiding a stopping tool. Such a stopping tool can in principle take any arbitrary shape and may include but is not limited to a pin, such as a cylindrical pin or a crescent shaped pin, a plate, a surface, a (rectangular) block, a square (block), a cylinder etc. and generally functions to prevent a surgical instrument from advancing beyond a planned or determined depth into the bone. More than one stopping tool can be used to stop the cutting instrument along a path.

Thus, where the stopping tool is a cylindrical pin, the sleeve element may comprise at least a cylindrical hole, the position and direction of which correspond to the pre-operatively planned trajectory. The diameter of the cylindrical hole will be at least the size of the diameter of the part of the pin, which is to be introduced in the bone.

In particular embodiments, where the stopping tool is a plate, a surface, a (rectangular) block, or a square (block), the sleeve element contain at least a (narrow) slot or flat surface, the position and orientation of which will correspond to the pre-operatively planned plane or surface along which the stopping tool is to be introduced into the bone. The dimensions of the slot or surface will be such to allow passage or suitable guidance of the plate, a surface, a (rectangular) block, or a square (block) that is used.

Accordingly, in particular embodiments, the sleeve element includes a cannula or channel through which at least part of a stopping tool may be passed to engage the bone at the desired location.

Typically, the sleeve element is connected via the bridge element (as further described herein) to the connecting element (as described herein) of the stopping tool guide in a manner such that a stopping tool which is passed through the sleeve element can engage the bone at a desired location and in a pre-operatively planned direction. This pre-operatively planned trajectory of the stopping tool is also referred to herein as direction y. Accordingly, if the stopping tool is a stopping pin, direction y of the sleeve element corresponds to the direction in which said pin is guided through the sleeve element into the bone. Alternatively, where the stopping tool is a plate, a surface, a block etc. direction y of the sleeve element corresponds to the direction of the plane along which said plate, a surface, a block etc. is guided through the sleeve element into the bone. The sleeve element can be positioned in any direction with regard to the central longitudinal axis of the bone as long as it provides access to a stopping tool for reaching the bone at the desired location. In particular embodiments of the invention, orientation direction y can also be considered to be the direction of the central axis of the sleeve element.

The pre-operatively planned trajectory of the stopping tool or the orientation direction y of the sleeve element of the stopping tool guide according to the present invention is such that it can interfere (i.e. stop) the surgical instrument when guided by the surgical guide. Thus it is envisaged that the direction of the sleeve is such that it crosses the plane in which the surgical tool is guided. In particular embodiments, the orientation axis of the sleeve is such that it lies within the plane along which the cutting instrument is guided by the guiding component of the surgical guide and crosses diagonally in that plane. Alternatively it can be envisaged that it crosses perpendicularly to that plane such that a stopping tool presented therein (either alone or in combination with a second stopping tool guide) interferes with the trajectory of the surgical tool in the guide. Indeed, the sleeve element guides a stopping tool in an orientation direction y, which stopping tool serves to prevent a surgical instrument (i.e. a cutting instrument) from advancing beyond a planned or determined depth into the bone. In particular embodiments, in order to achieve this function of the stopping tool, the direction in which this tool is guided, i.e. orientation direction y, is in the cutting plane of the cutting instrument, i.e. the plane along which the cutting instrument is guided by the guiding component of the surgical guide. Alternatively however, it can be in a different plane, more particularly one that is perpendicular to the cutting plane of the instrument.

In particular embodiments of the invention, the sleeve element of the stopping tool guide, is not in contact with the bone or the surgical guide when the stopping tool guide is in use and thus a gap or space exists between the guide and/or the bone and the sleeve element. The existence of such a gap or space is ensured by the presence and nature of the bridging element of the stopping tool guide which forms an interconnection with the connecting elements of the stopping tool guides, but at the same time ensures that the sleeve is maintained at a certain distance from the bone and extends away from the surgical guiding tool. Accordingly, the stopping tool guides of the present invention only make contact with the surgical guides of the invention via the connecting elements of the stopping tool guides. The stopping tool guides of the present invention and combinations thereof with surgical guides allow for minimal invasive surgery as there is no need for an additional surgical intervention to define the position of the stopping tool is eliminated. In particular embodiments, where the stopping tool is a pin, percutaneous pin insertion is used to insert the pin into the bone, allowing for minimal invasive surgery.

Where it is envisaged that the bridge element is such that the sleeve element is not in contact with the bone or the surgical guide, the surgical guide can also contain an insert feature into which the stopping tool will fit, allowing further guidance of the tool as it is placed in the bone, thus positioning it more accurately. In particular embodiments, this insert feature may be in contact with the bone.

Additionally, the sleeve element may also be reversibly detachable from the stopping tool guide. Where the connection is rigid, mechanisms such as a snap fit feature may be used to provide a stable connection between the guides or the elements of a guide. In particular embodiments, the sleeve element comprises a reversibly detachable sleeve insertion element, which is reversibly inserted into the sleeve element in orientation direction y. In other particular embodiments, a sleeve element further can be provided with a drill sleeve for guiding a drilling instrument, which can be used to pre-drill the pre-defined trajectory along which the stopping tool is to be inserted into the bone.

The stopping tool guides according to the present invention that can be used in combination with surgical guides thus typically comprise a bridge element interconnecting the connecting element (as described herein) with the sleeve element (as described herein) of the stopping tool guides. The bridge element can be reversibly connectable to the connecting element and sleeve element of the stopping tool guide or can be an integral part of said stopping tool guide.

The bridge element comprised in the stopping tool guides of the present invention is designed to connect the stopping tool guide and the surgical guide, while allowing the stopping tool guide to be positioned at a desired orientation with regard to the orientation of the guiding features in the surgical guide. The bridge can be tailored to a specific size or can be a generic part in one or more standard sizes.

When the stopping tool guide is placed on a surgical guide on a bone, the bridge element itself is neither in contact with the bone nor in contact with the surgical guide and can be located either above or on top of the skin or underneath the skin. This is ensured by the shape of the bridge which is adjusted to ensure some distance with the surface of the bone. In view of this distance from the bone in combination with the fact that it extends from the connecting element (for the connecting to the surgical guide) which is positioned on one part of the bone, to the sleeve which is positioned near an opposing side of the bone, it is referred to as a “bridge”. In those particular embodiments, where it is envisaged that the bridge element is positioned above or on top of the skin, its optimal size, position and/or orientation can be determined using soft tissue information from the patient's images. In particular embodiments, the bridge element is adapted to reduce or eliminate stress caused by the expected loads during surgery. This prevents deformation of the bridge during surgery. In further embodiments, the bridge is designed such that deformation is especially minimized along the direction of the stopping tool. In certain embodiments, the bridge element has an open and/or hollow structure. This reduces the weight of the bridge, which prevents destabilization of the surgical guide by the weight of the stopping tool guide. The open structure further allows minimizing the amount of material required for manufacturing the stopping tool guides.

The bridge element is (optionally reversibly) attached by one end to the connecting element (as described herein) of the stopping tool guide. In particular embodiments it is envisaged that the bridge is (optionally reversibly directly attached to (the connecting element (as described herein) of) the surgical guide.

As detailed above, the surgical guide and the stopping tool guide of the invention are or can be interconnected by these connecting elements and can be reversibly detachable. In those particular embodiments, where the surgical guide and the stopping tool guide are reversibly detachable or removable from each other, these connecting elements of the surgical guide and the stopping tool guide may provide for a locking mechanism, such as a clip-on, slide lock or snap-fit mechanism, a pinned system or the like by which they are interconnected or interlocked.

The bridging elements comprised in the stopping tool guides of the present invention are designed such that they either ensure a fixed distance (or angular difference) between the connection and the sleeve element of the stopping tool guide, or can be adjusted to ensure an appropriate angular difference between the connecting element and the sleeve element. In particular embodiments, the angular difference that is bridged by said bridge element between said connecting element (based on an orientation which is determined as detailed above) and said sleeve element is between 30° and 180°, such as between 30° and 180°, in particular between 40° and 170°, more particularly between 50° and 160°, such as between 60° and 150° or between 70° and 140°, such as for example between 75° and 120°, such as between 75° and 100°, such as about 80°, in particular about 85°, such as about 90° or about 95°.

In the present description of the bridging elements comprised in the stopping tool guides of the invention, reference is made to angles or to an angular difference to define the extent to which the bridging element bridges the bone with regard to the central longitudinal axis of the bone (i.e. to define the angular difference by which the bridging element interconnects said connecting element with said sleeve element of the stopping tool guides with regard to the central longitudinal axis of the bone). In particular embodiments, reference is made to the angle between orientation direction x of said connecting element and orientation direction y of said sleeve element. In this context, it will be understood to the skilled person, that in general, limited variations of the angles around the indicated values will not significantly impact the function of the structures of the stopping tool guides and can be considered as obvious alternatives of the values provided. Accordingly, where referring to an angle in the context of the present invention, unless specified, typically variations of +/−5° should be considered as equally envisaged. Similarly, in some embodiments variations of +10° or −10° may also be envisaged.

The bridge element comprised in the stopping tool guides of the present invention interconnects the connecting element of the stopping tool guide with the sleeve element of the stopping tool guide. The bridge element can be designed to be patient-specific, i.e. ensuring a specific angle between the connecting element and the sleeve element of the stopping tool. More particularly, the guides of the present invention are manufactured in one piece and based on patient-specific information. However, it is also envisaged that the bridge element can be a modular element, of which the length can be adjusted. Additionally or alternatively, the bridge element can be a generic element, which can be reversibly connected to the connecting element on the one hand and the sleeve element on the other. Where the bridge element is made of a material such as steel, the bridge is a part with lower tolerance.

In certain particular embodiments, the angular difference that is (or can be) bridged by said bridging element corresponds to the angle between orientation direction x (as described herein) of the connecting element and orientation direction y (as described herein) of the sleeve element.

Indeed, in particular embodiments of the present invention, the connecting element is said to have an orientation direction x. In such embodiments, orientation direction x can be but is not limited to the direction in which the stopping tool guide is positioned on the surgical guide; this is particularly the case where the stopping tool guide is placed onto the surgical guide in a direction that is not parallel (but for example perpendicular or substantially perpendicular) to the central longitudinal axis of the bone, e.g. by using a clip-on mechanism. However, in certain other particular embodiments, where the stopping tool guide is placed onto the surgical guide in a direction that is parallel or substantially parallel to the central longitudinal axis of the bone, e.g. by a slide lock mechanism, orientation direction x is different from the direction in which the stopping tool guide is placed or connected to the surgical guide. In further particular embodiments, orientation direction x corresponds to the central (i.e. symmetry or rotational axis) of the connecting element, e.g. when the connecting element has a cylindrical shape, orientation direction x corresponds to the central longitudinal axis of the cylindrical shape of the connecting element.

In certain particular embodiments, the pre-operatively planned trajectory of the stopping tool is also referred to as direction y. In particular embodiments of the invention, orientation direction y can also be considered to be the direction of the central axis of the sleeve element.

Accordingly, in particular embodiments of the present invention the angle between orientation direction x of the connecting element and orientation direction y of the sleeve element of the stopping tool guides of the present invention is between 30° and 180°, in particular between 40° and 170°, more particularly between 50° and 160°, such as between 60° and 150° or between 70° and 140°, such as for example between 75° and 120°, such as between 75° and 100°, such as about 80°, in particular about 85°, such as about 90° or about 95°.

In particular embodiments, the bridge has a semi-circular shape, more particularly an arch-like shape.

As detailed above, in particular embodiments the bridge is positioned such that the sleeve element of the stopping tool guide connected thereto, is not in contact with the bone or the surgical guide when the combination of surgical guiding instrument and stopping guide is placed on the bone, and thus a gap or space exists between the bone and the sleeve element. In further particular embodiments, the bridge may extend along the circumference of the bone at a limited distance in order to avoid that it is a visual and/or practical hindrance to the surgeon. Thus in particular embodiments, the position of the bridge is such that, when the stopping tool guide is positioned on the surgical guide, the bridge extends around the bone at a distance between 2 and 15 cm from the surface of the bone.

A further aspect of the present invention provides methods for obtaining a combination of a stopping tool guide and a surgical guide according to the present invention.

The methods for obtaining a combination of a stopping tool guide and a surgical guide, comprise creating a pre-operative plan, based on one or more images of the bone of a patient to determine the desired cutting path of the bone along at least one plane. Accordingly, the methods comprise the step of determining the appropriate position with regard to the bone of the guiding component for guiding a cutting instrument along a plane of said surgical guide, based on the pre-operative planning of the desired path of said cutting instrument into the bone.

Images of the bone suitable for use in the methods of the present invention can be obtained by the skilled person. Digital patient-specific image information can be provided by any suitable means known in the art, such as for example a computer tomography (CT) scanner, a magnetic resonance imaging (MRI) scanner or an ultrasound scanner. A summary of medical imaging has been described in “Fundamentals of Medical imaging”, by P. Suetens, Cambridge University Press, 2002.

In particular embodiments, the methods for providing the stopping tool guides and combinations thereof with surgical guides described herein also comprise obtaining an image of the bone.

After the appropriate position of the guiding component has been determined, the position and critical dimension of a stopping tool that will act as a physical stop to the cutting instrument when it has reached the desired position along the cutting path, is determined. Accordingly, a further step of the methods comprises determining the appropriate position of a stopping tool in the bone, based on the pre-operative planning of the desired path of said stopping tool into the bone.

In particular embodiments, the steps of determining the position of the guiding component and the stopping tool is performed based on the pre-operative planning of the desired paths. The orientation of the cutting instrument and the stopping tool has to be such that they are guided in the predetermined direction. Pre-operative planning of the intervention by the treating physician makes it possible to determine the required path of the surgical instrument, and accordingly, the required orientation of the guiding component of the surgical guide and/or the sleeve element of the stopping tool guide. The pre-operative planning of the surgical intervention is done using suitable dedicated software, based on suitable medical images (of which CT, MRI, are examples), taking into account factors like bone quality and proximity to nerve bundles/blood vessels or other anatomically sensitive objects. In particular embodiments, the critical dimensions of the stopping tool are based on crack propagation analysis. To plan and simulate the intervention, preoperative images are imported into a computer workstation running 3D software. These images are manipulated as 3D volumes. The result is a computer simulation of the intervention, which outputs a planning containing the information necessary for adapting the orientation of the guiding component of the surgical guide and/or the sleeve element of the stopping tool guide.

Having defined the critical dimensions, for example (but not limiting to) position, orientation, depth and diameter of the trajectories or paths of the stopping tools and the cutting instruments associated to them, this information can then be transferred to a surgical guide and a stopping tool guide, which perfectly fit when placed on the bone.

Thus, thereafter, a surgical guide is designed to be placed on the bone using at least one surface feature and to guide the cutting instrument along the pre-determined cutting path. Furthermore, a stopping tool guide is designed containing a sleeve element that will guide the stopping tool to be accurately positioned as such. Accordingly, step (d) of the methods of manufacturing according to the present invention comprises designing the combination of said stopping tool guide and said surgical guide based thereon such that:
said guiding component for guiding a cutting instrument, upon placement of the surgical guide on the bone, is positioned as determined in step (b);
said sleeve element of said stopping tool guide ensures the correct orientation of the stopping tool upon placement of the stopping tool guide on the bone;
said bridge element ensures that said stopping tool can be connected to said surgical guide such that the angular difference that is bridged by said bridge element between said connecting element and said sleeve element is between 30° and 180°, and
said bridge element ensures that said sleeve element is not in contact with said surgical guide and/or with the bone upon placement of said combination of said stopping tool guide and said surgical guide onto the bone.

In particular embodiments, a further step of the methods for providing the stopping tools and combinations according to the invention comprises preparing or manufacturing the stopping tool guide and the surgical guide. In particular embodiments, the step of preparing such surgical tools comprises producing and assembling the different parts of stopping tool guide and the surgical guide according to the invention, i.e. producing a surgical guide comprising a guiding component (as described herein), a surface structure, and a connecting element, and producing a surgical stopping tool guide comprising a connecting element, a sleeve element, and a bridge element interconnecting said connecting element with said sleeve element,

wherein the angular difference that is bridged by said bridge element between said connecting element and said sleeve element is between 30° and 180°, and

wherein said orientation direction y of said sleeve element lies within said plane for guiding said cutting instrument.

In particular embodiments, where one or more of the components of the guiding tools of the present invention are made of one piece, the step of “assembling” may correspond to combining the relevant information for the combined manufacture of the pieces involved.

In particular embodiments the step of preparing the surgical guiding tool comprises the production of one or more elements by rapid prototyping. In further particular embodiments, the step of preparing the surgical guiding tool comprises the production of the surgical guiding tool as defined in the present invention, by rapid prototyping.

Rapid prototyping includes all techniques whereby an object is built layer by layer or point per point by adding or hardening material (also called free-form manufacturing). The best known techniques of this type are stereolithography and related techniques, whereby for example a basin with liquid synthetic material is selectively cured layer by layer by means of a computer-controlled electromagnetic beam; selective laser sintering, whereby powder particles are sintered by means of an electromagnetic beam or are welded together according to a specific pattern; fused deposition modeling, whereby a synthetic material is fused and is stacked according to a line pattern; laminated object manufacturing, whereby layers of adhesive-coated paper, plastic, or metal laminates are successively glued together and cut to shape with a knife or laser cutter; or electron beam melting, whereby metal powder is melted layer per layer with an electron beam in a high vacuum.

In a particular embodiment, Rapid Prototyping and Manufacturing (RP&M) techniques, are used for manufacturing the surgical guiding tool. Rapid Prototyping and Manufacturing (RP&M) can be defined as a group of techniques used to quickly fabricate a scale model of an object typically using three-dimensional (3-D) computer aided design (CAD) data of the object. Currently, a multitude of Rapid Prototyping techniques is available, including stereo lithography (SLA), Selective Laser Sintering (SLS), Fused Deposition Modeling (FDM), foil-based techniques, etc.

A common feature of these techniques is that objects are typically built layer by layer. Stereo lithography, presently the most common RP&M technique, utilizes a vat of liquid photopolymer “resin” to build an object a layer at a time. On each layer, an electromagnetic ray, e.g. one or several laser beams which are computer-controlled, traces a specific pattern on the surface of the liquid resin that is defined by the two-dimensional cross-sections of the object to be formed. Exposure to the electromagnetic ray cures, or, solidifies the pattern traced on the resin and adheres it to the layer below. After a coat had been polymerized, the platform descends by a single layer thickness and a subsequent layer pattern is traced, adhering to the previous layer. A complete 3-D object is formed by this process.

Selective laser sintering (SLS) uses a high power laser or another focused heat source to sinter or weld small particles of plastic, metal, or ceramic powders into a mass representing the 3-dimensional object to be formed.

Fused deposition modeling (FDM) and related techniques make use of a temporary transition from a solid material to a liquid state, usually due to heating. The material is driven through an extrusion nozzle in a controlled way and deposited in the required place as described among others in U.S. Pat. No. 5,141,680.

Foil-based techniques fix coats to one another by means of gluing or photo polymerization or other techniques and cut the object from these coats or polymerize the object. Such a technique is described in U.S. Pat. No. 5,192,539.

Typically RP&M techniques start from a digital representation of the 3-D object to be formed. Generally, the digital is sliced into a series of cross-sectional layers which can be overlaid to form the object as a whole. The RP&M apparatus uses this data for building the object on a layer-by-layer basis. The cross-sectional data representing the layer data of the 3-D object may be generated using a computer system and computer aided design and manufacturing (CAD/CAM) software.

A selective laser sintering (SLS) apparatus is particularly preferred for the manufacture of the guiding template from a computer model. It should be understood however, that various types of rapid manufacturing and tooling may be used for accurately fabricating these surgical templates including, but not limited to, stereolithography (SLA), Fused Deposition Modeling (FDM) or milling.

The surgical guides and stopping tool guides of the invention (or parts thereof) may be manufactured in different materials. Typically, only materials that are biocompatible (e.g. USP class VI compatible) with the human body are taken into account. Preferably the surgical template is formed from a heat-tolerable material allowing it to tolerate high-temperature sterilization. In the case SLS is used as a RP&M technique, the surgical template may be fabricated from a polyamide such as PA 2200 as supplied by EOS, Munich, Germany or any other material known by those skilled in the art may also be used.

In a further aspect, the present invention provides methods for accurately performing surgical osteotomy on a bone, which method comprises a step (a) of providing a combination of a stopping tool guide and a surgical guide according to the invention. This can for example be done using the methods for manufacturing the guides and combinations of the invention as described above.

A further step (b) in the methods for performing a surgical osteotomy using a stopping tool guide and a surgical guide according to the invention comprises securing the combination of the stopping tool guide and the surgical guide onto the bone by aligning it in the desired direction with regard to the anatomical surface on the bone.

The methods for performing a surgical osteotomy using a stopping tool guide and a surgical guide according to the invention further comprise, after securing the combination of the stopping tool guide and the surgical guide onto the bone the step (c) of introducing a stopping tool through the sleeve element of the stopping tool guide into the bone. Thus, in the methods according to the present invention, the sleeve element is designed to be positioned on a unique and stable position, specific for a patient and intended to be used for guiding a stopping tool, according to a prior determined position, direction and/or path and/or depth. At least part of the stopping tool is therefore introduced through a hole, a cannula, a channel or a slot or along a flat surface to engage the bone at the desired location.

A further step (d) in the methods for performing a surgical osteotomy comprises introducing a cutting instrument through the at least one guiding component of the surgical guide, whereby the cutting instrument is stopped at a specifically pre-determined position by the stopping tool.

The invention will now be illustrated by the following, non-limiting illustrations of particular embodiments of the invention.

### EXAMPLES

### Example 1

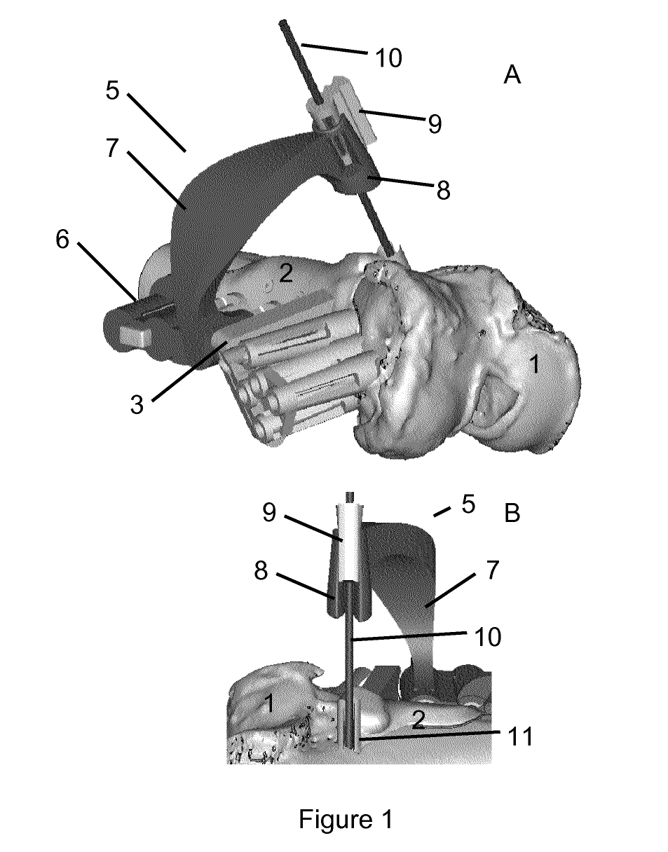

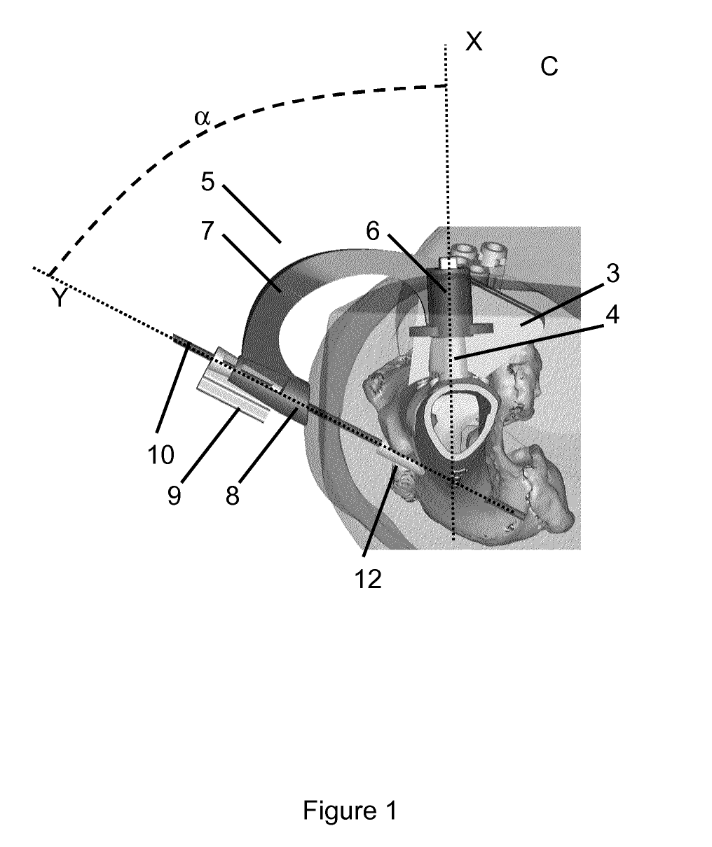

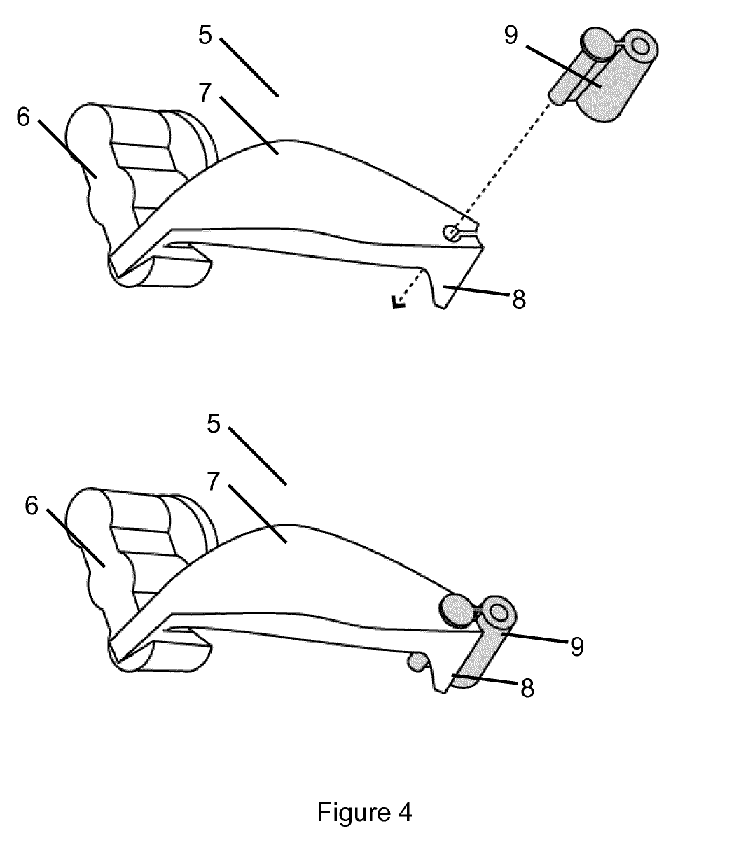

This example describes a specific but non-limiting embodiment of the stopping tool guide and a surgical guide according to the present invention. As shown in FIG. 1 stopping tool guide and a surgical guide are provided an anatomical part (1) of a patient, the lower extremity of the femur in the present case. The surgical guide (2) is provided with a guiding component for guiding a cutting instrument along a plane (3), a surface structure which is may optionally be patient-specific (not visible) and a connecting element (4) for connecting the surgical guide to a stopping tool guide (5). The stopping tool guide (5) comprises a connecting element (6) for connecting the stopping tool guide (5) to the connecting element (4) of the surgical guide (2), a sleeve element (8) for guiding the stopping tool (10) in a specific orientation, and a bridge element (7) interconnecting the connecting element (6) with the sleeve element (8). As shown in FIG. 1 the sleeve element (8) comprises a sleeve insertion element (9) for guiding the stopping tool (10) in a specific orientation. An alternative form of the sleeve insertion element (9) is also provided in FIG. 4.

FIGS. 1b and 1c show the same combination of stopping tool guide and a surgical guide from different angular views. Optionally the surgical guide may further comprise an insert feature (12) on the surface structure for guiding the stopping tool (10) into the bone.

The orientation direction of the sleeve element (8) is attributed the Y direction whereas orientation direction of the connection between the stopping tool guide (5) and the surgical guide (2) is attributed the X direction.

The combination of stopping tool guide (5) and surgical guide (2) according to the present invention further provides that the angular difference (a) that is bridged by the bridge element (7) between the connecting element (6) and the sleeve element (8) is between 30° and 180°.

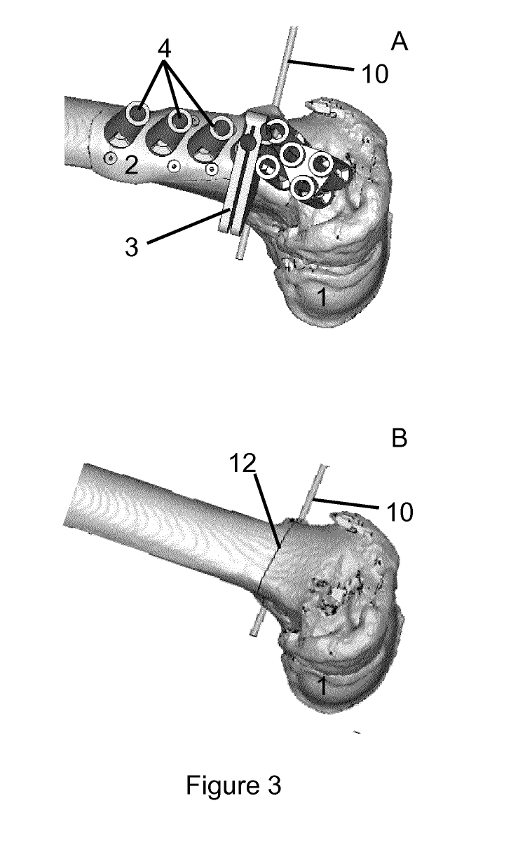

The combination of stopping tool guide (5) and surgical guide (2) according to the present invention as provided here allow the introduction of a stopping tool on a pre defined and possibly patient-specific position without the need to perform even more complicated surgical steps, thereby allowing to perform a surgical osteotomy by introducing a cutting instrument through the guiding component of the surgical guide, whereby the cutting instrument is stopped by the stopping tool. Therefore as shown in FIG. 3, after insertion of the stopping tool (10) the stopping tool guide (5) is optionally removed from the surgical guide (2) and using the guiding component for guiding a cutting instrument along a plane (3) osteotomy is performed, thereby cutting the bone partially until the cutting action is stopped by the stopping tool (10) resulting in a partial bone osteotomy (12).

### Example 2

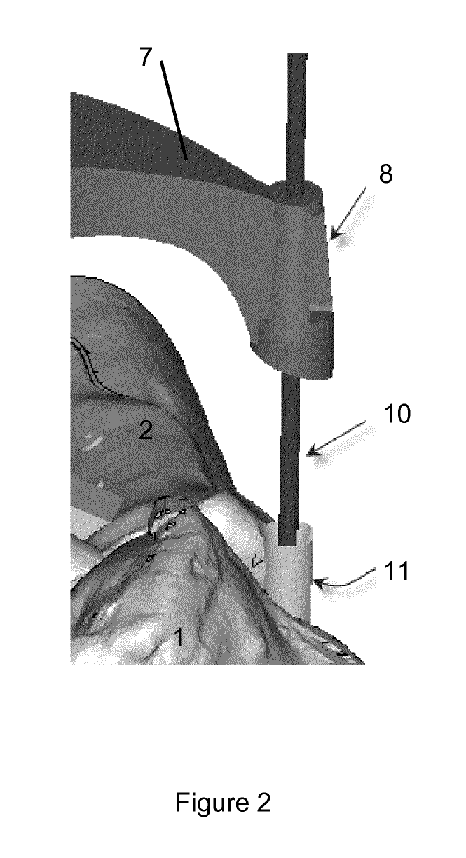

This example describes a specific but non-limiting embodiment of the stopping tool guide and a surgical guide according to the present invention. Whereas the sleeve element (8) in FIG. 1 comprises a sleeve insertion element (9) for guiding the stopping tool (10), an alternative embodiment is provided as shown in FIG. 2 wherein the sleeve element (8) does not require an additional sleeve insertion element (9), but the sleeve element (8) itself allows the guidance of the stopping tool (10) in a specific orientation direction.

### Example 3

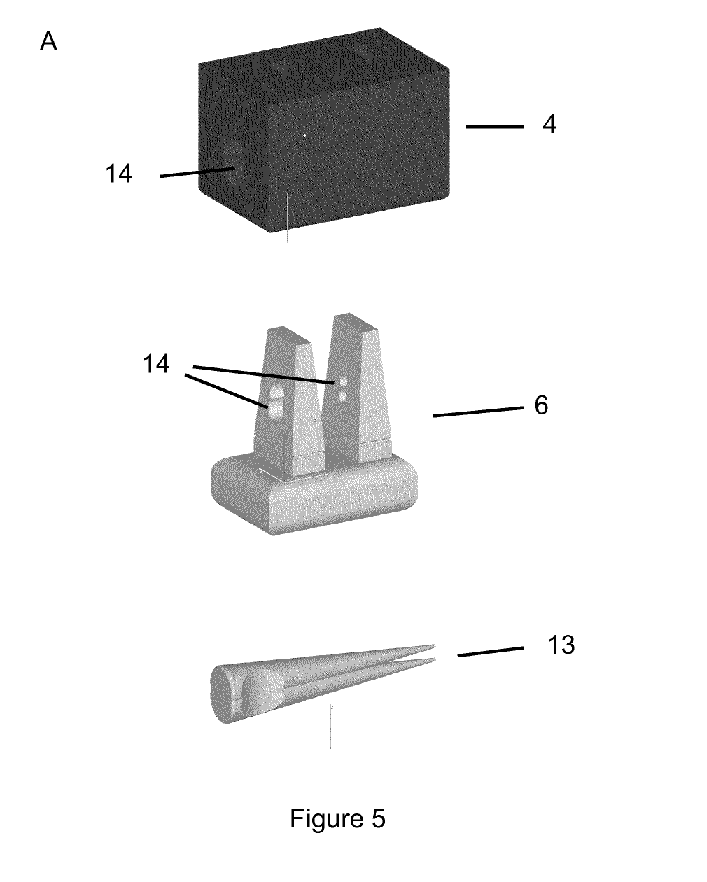

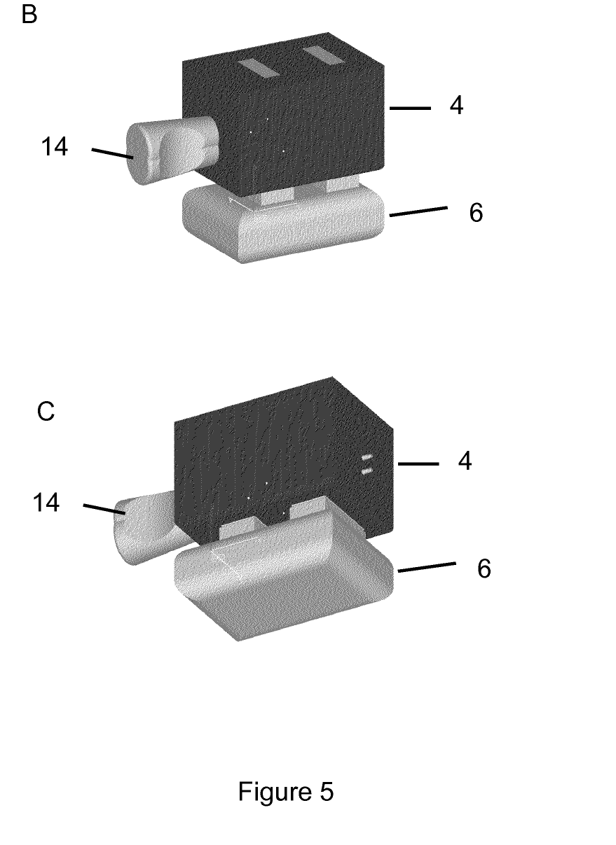

FIG. 5 A illustrates two connecting elements (4, 6) according to a particular embodiment of the present invention, which can be used in combination for connecting a stopping tool guide to a surgical guide (not shown).

Connecting element (6) is part of a surgical guide as described herein (not shown) and connecting element (4) is part of a stopping tool guide as described herein (not shown), or vice versa. The connecting elements allow connecting the stopping tool guide and the surgical guide by inserting the protrusions of connecting element (6) into matching recesses of connecting element (4). Each of the connecting elements further comprises one or more holes (13), allowing insertion of a (dual) locking pin (14). Insertion of the locking pin after connection of the connecting elements as shown in FIG. 5 B (top front left view) and FIG. 5 B (bottom front right view), thereby fixing the relative position of the connecting elements. The shape of the broad end of the locking pin provides additional grip, which facilitates pulling the pin out when the surgeon decides to detach the stopping tool guide from the surgical guide.

### Example 4

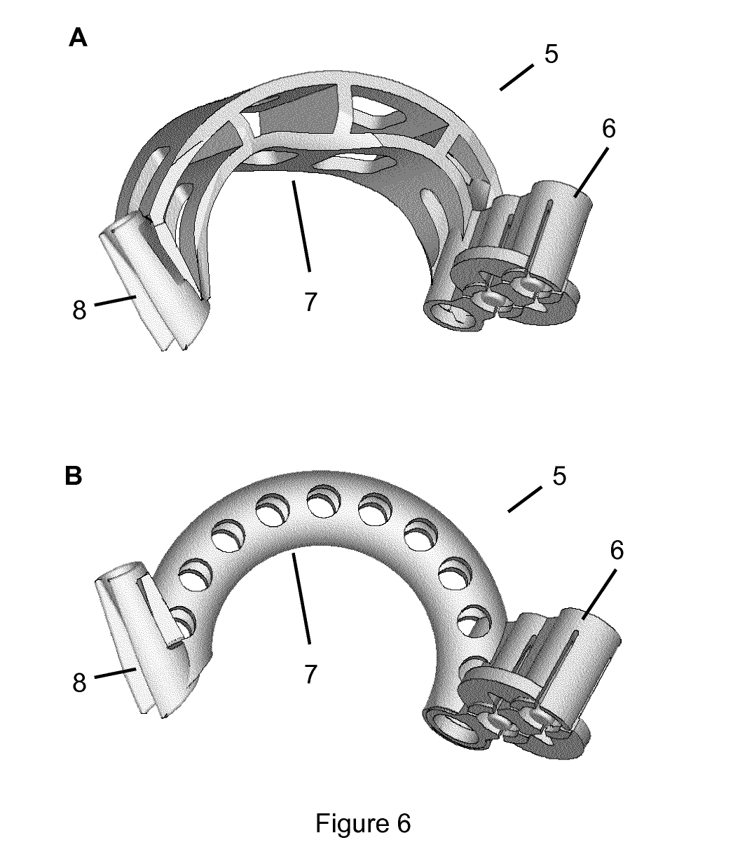

FIGS. 6A and B illustrate specific but non-limiting embodiments of stopping tool guides (5) according to the present invention. Each stopping tool guide comprises a connecting element (6) for connecting the stopping tool guide to a connecting element of a surgical guide (not shown), a bridge element (7) and a sleeve element (8) suitable for receiving a sleeve insertion element (not shown).

The bridge elements (7) have an open structure, which provides an enhanced resistance and rigidity against the expected loads during surgery, while minimizing the weight of the bridge elements, and minimizing the amount of material required for manufacturing the stopping tool guides.

1. A combination of a stopping tool guide and a surgical guide for a cutting instrument for cutting a bone of a patient,
wherein said surgical guide comprises:
(i) at least one guiding component for guiding said cutting instrument along a plane,
(ii) at least one surface structure for contacting said patient's bone, and
(iii) a connecting element for connecting said surgical guide to said stopping tool guide;
and wherein said stopping tool guide comprises:
(i) a connecting element for connecting the stopping tool guide to said connecting element of said surgical guide, whereby said stopping tool guide is reversibly detachable from said surgical guide,
(ii) at least one sleeve element for guiding said stopping tool in orientation direction y,
and
(iii) a bridge element interconnecting said connecting element of said stopping tool with said sleeve element,
wherein the angular difference that is bridged by said bridge element between said connecting element and said sleeve element is between 30° and 180°, and
wherein said orientation direction y of said sleeve element crosses the plane of said cutting instrument, such that said stopping tool functions to prevent said cutting instrument, when inserted into the guide component, from advancing beyond a planned or determined depth.

2. The combination of a stopping tool guide and a surgical guide according to claim 1, wherein the bridge element is a generic element, which can be reversibly connected to the connecting element and the sleeve element.

3. The combination of a stopping tool guide and a surgical guide according to claim 1, wherein said bridge element ensures that said sleeve element is not in contact with said surgical guide and/or with the bone upon placement of said combination of said stopping tool guide and said surgical guide onto the bone.

4. The combination of a stopping tool guide and a surgical guide according to claim 1, wherein said sleeve element is reversibly detachable from said stopping tool guide.

5. The combination of a stopping tool guide and a surgical guide according to claim 1, wherein said sleeve element comprises a reversibly detachable sleeve insertion element, which is reversibly inserted into said sleeve element in said direction y.

6. The combination of a stopping tool guide and a surgical guide according to claim 4, wherein said sleeve element further can be provided with a drill sleeve for guiding a drilling instrument.

7. The combination of a stopping tool guide and a surgical guide according to claim 1, wherein said surgical guide further comprises an insert feature on said surface structure for guiding said stopping tool into the bone in orientation direction y.

8. The combination of a stopping tool guide and a surgical guide according to claim 1, wherein said surgical guide is a patient-specific surgical guide.

9. A stopping tool guide comprising:
(i) a connecting element for connecting the stopping tool guide to a surgical guide,
(ii) a sleeve element for guiding said stopping tool in orientation direction y,
and
(iii) a bridge element interconnecting said connecting element with said sleeve element,
wherein the angular difference that is bridged by said bridge element between said connecting element and said sleeve element is between 30° and 180°.

10. The stopping tool guide according to claim 9, wherein said bridge element ensures that said sleeve element of said stopping tool guide is not in contact with said surgical guide and/or with the bone upon placement of the said stopping tool guide onto the a surgical guide or onto a bone.

11. A method for providing a combination of a stopping tool guide and a surgical guide according to claim 1, said method comprising:
(a) determining the appropriate position with regard to the bone of said guiding component for guiding a cutting instrument along a plane of said surgical guide, based on an image of the bone and the pre-operative planning of the desired path of said cutting instrument into the bone;
(b) determining the appropriate position of a stopping tool in the bone, based on the pre-operative planning of the desired path of said cutting instrument into the bone; and
(c) designing the combination of said stopping tool guide and said surgical guide based thereon such that:
said guiding component for guiding a cutting instrument, upon placement of the surgical guide on the bone, is positioned as determined in step (b), and
said sleeve element of said stopping tool guide ensures the correct orientation of the stopping tool upon placement of the stopping tool guide on the bone,
said bridge element ensures that said stopping tool can be connected to said surgical guide such that the angular difference that is bridged by said bridge element between said connecting element and said sleeve element is between 30° and 180°,
and ensures that said sleeve element is not in contact with said surgical guide and/or with the bone upon placement of said combination of said stopping tool guide and said surgical guide onto the bone, and

12. The method of claim 11, which further comprises manufacturing said stopping tool guide and said surgical guide.

13. The method of claim 12, wherein said step of manufacturing is ensured by additive manufacturing.

14. A method for performing accurate surgical osteotomy on a bone, which method comprises:
(a) providing a combination of a stopping tool guide and a surgical guide as defined in claim 1,
(b) securing said combination of said stopping tool guide and said surgical guide onto the bone by aligning it in the desired direction with regard to the anatomical surface on the bone,
(c) introducing a stopping tool through the sleeve element of said stopping tool guide into the bone, and
(d) performing a surgical osteotomy by introducing a cutting instrument through said at least one guiding component of said surgical guide, whereby said cutting instrument is stopped by said stopping tool.
